(19) United States
(12) Patent Application Publication
(10) Pub. No.: US 2015/0044183 A1
(43) Pub. Date: Feb. 12, 2015

(54) STEM CELL BASED ANTI-CANCER COMPOSITIONS AND METHODS

(71) Applicant: PARCELL LABORATORIES, LLC, Natick, MA (US)

(72) Inventors: Keith D. Crawford, Westwood, MA (US); John Garvey, Londonderry, NH (US)

(21) Appl. No.: 14/453,937

(22) Filed: Aug. 7, 2014

Related U.S. Application Data
(60) Provisional application No. 61/863,243, filed on Aug. 7, 2013.

(51) Int. Cl. A61K 35/12; A61K 33/22; A61K 33/24

(52) U.S. Cl. CPC A61K 35/12; A61K 33/24; A61K 33/22
USPC 424/93.7; 435/366

(57) ABSTRACT

The present invention provides compositions and methods for target radiotherapeutic treatment of cancer. Freshly isolated or culture expanded ELA stem cells are loaded with at least one of a diagnostic agent and a therapeutic agent, and the stem cells are then introduced into a subject. The loaded ELA stem cells migrate to location of cancer and form the basis both of diagnostic imaging and of directed Neutron Capture Therapy. Treatment of a subject may include ELA stem cell-based NCT radiotherapy as an adjunct to traditional therapies. An ELA stem cell-based diagnostic preparation can be used directly, or as a further addition to current imaging agents and methods.

### RELATED APPLICATION

This application claims the benefit of U.S. provisional application Ser. No. 61/863,243 filed Aug. 7, 2013 entitled, “Stem cell based anti-cancer compositions and methods”, which is incorporated by reference herein in its entirety.

### FIELD OF INVENTION

The present invention relates to adult stem cell compositions and methods for diagnosis and for treatment of cancers. More specifically, the present invention relates to the use of ELA™ human derived adult stem cell compositions to identify and kill neoplastic tissues, ameliorate inflammation, reduce pain, and regenerate the tissues to a healthier state.

### SUMMARY

An aspect of the invention provides a cell-based preparation including: a population of loaded ELA stem cells loaded with a diagnostic agent and/or a therapeutic agent. The term “loaded” in general refers to the presence of the diagnostic agent and/or the therapeutic agent in the ELA stem cells, such that the ELA cells may be imaged/tracked using the diagnostic agent or may be effective for delivery of the therapeutic agent to a cancer or tumor. For example, a sample of ELA cells is made to contain, made to carry, incubated, or cultured with the diagnostic agent and/or the therapeutic agent such that all or a portion of the ELA stem cells contain the diagnostic agent and/or the therapeutic agent. In various embodiments, the cells contain the diagnostic agent and/or the therapeutic agent; however, the cells do not have the activity necessary to be imaged or effectively treat a tissue. In various embodiments, the cells not having the diagnostic agent and/or the therapeutic agent or not having the necessary activity to image or treat the tissue are referred to as “unloaded” ELA stem cells.

In various embodiments, the diagnostic agent includes a gadolinium, for example Gadolinium-157. In various embodiment of the preparation, the therapeutic agent includes Gadolinium-157 and/or Boron-10.

The cell-based preparation in various embodiments contains from 10² to 10⁸ loaded ELA stem cells in a volume for example of about 0.5 to about 10 ml of a pharmaceutically acceptable solution. In various embodiments, the cell-based preparation further includes a perfluorate agent. For example, the perfluorate agent includes a perfluorate gas.

In various embodiments, the diagnostic agent includes an imaging agent or contrast agent. For example, the imaging agent or contrast agent is used in positron emissions tomography (PET), computer assisted tomography (CAT), single photon emission computerized tomography, x-ray imaging, fluoroscopy, and magnetic resonance imaging (MRI). In various embodiments the imaging agent or the contrast agents includes gadolinium chelates, as well as materials (e.g. nanoparticles and beads) that contain iron, magnesium, manganese, copper, and chromium. For example, the materials are useful for CAT and x-ray imaging, or include barium, thallium, or iodine-based materials. In various embodiments, the imaging agent or the contrast agent includes a radioactive compound or radioactive sugar molecule, for example 18F-fluorodeoxyglucose (FDG). In various embodiments, the imaging agent or the contrast agent includes at least one of: a nuclear imaging agent, an X-ray imaging agent, a MRI imaging agent, or a radioactive contrast agent. In various embodiments, the imaging agent or the contrast agent includes a paramagnetic contrast reagent, for example Molday ION Rhodamine B imaging agent (BioPal Inc.; Worcester, Mass.).

In various embodiments, the therapeutic agent is selected from: anti-coagulant, anti-tumor, anti-viral, anti-bacterial, anti-mycobacterial, anti-fungal, and anti-proliferative. In various embodiments, the therapeutic agent is selected from the group of: adriamycin, methotrexate, cisplatin, paclitaxel, doxorubicin, vinblastine, vincristine, BCNU (1,3-bis(2-chloroethyl)-1-nitrosourea), camptosar, 5-fluorouracil, Gleevac, Velcade (PS-341), ZD0473, and oncovine.

In various embodiments of the preparation, the therapeutic agent is a carbohydrate, a drug, a protein (e.g., an antibody), a peptide, a genetic material (e.g., DNA or RNA), or a polymer. In various embodiments, the ELA stem cells are administered an agent that induces the ELA stem cells to express the therapeutic agent.

For example, the drug is an anti-cancer agents that is administered with the ELA stem cells as described herein, using the methods and kits herein. Such drugs include for example pamidronate (Aredia); anastrozole (Arimidex); exemestane (Aromasin); bleomycin (Blenoxane); irinotecan (Camptosar); leucovorin; daunorubicin; cytaravine (CepoCyt); epirubicin (Ellence); etopos09901 ide (Etopophos); toremifene (Fareston); letrozole (Femara); gemcitabine (Gemzar); imatinib (Gleevac); topotecan (Hycamtin); tamoxifen (Nolvadex); paclitaxel (Taxol); docetaxel (Taxotere); capecitabine (Xeloda); temoxolomide (Temodar); nitrosourea; procarbazine (Matulane); valrubicin (Valstar); and goserelin (Zoladex).

In various embodiments of the preparation, the ratio of loaded ELA stem cells to unloaded ELA stem cells in the preparation is greater than about 2:1, about 3:1, or about 4:1. In various embodiments of the preparation, the ratio of loaded ELA stem cells to unloaded ELA stem cells in the preparation is greater than about 5:1.

An aspect of the invention provides a method of treating a subject having a cancer or a neoplasm, the method including: introducing to the subject a quantity of the cell-based preparation containing ELA stem cells, for example any of the preparations described herein, which targets to the neoplasm, optionally imaging the subject, then exposing the subject to a neutron source of greater than 0.5 electron volt (eV) to about 10 keV, thereby treating the neoplasm of the subject by radiotherapy. For example, the cancer or the neoplasm is benign, pre-malignant, or malignant.

In various embodiments of the method, the cancer is at least one type or kind selected from the group of a melanoma, a colon carcinoma, pancreatic, lymphoma, leukemia, brain such as glioma, lung, esophageal, a mammary/breast, prostate cancer, head, mouth, eye, bone, neck cancer, ovarian cancer, kidney, and liver. In various embodiments of the method, introducing the preparation includes contacting cells or a tissue by at least one route selected from the group of: parenteral, intravenous, intramuscular, intraperitoneal, intrapulmonary, intravaginal, rectal, oral, topical, sublingual, intranasal, ocular, transdermal, and subcutaneous.

The method in various embodiments further includes the step of obtaining from the subject a population of ELA cells, isolating the ELA cells and extending the ELA cells by culture prior to loading and administration to the subject. In various embodiments, obtaining the population of ELA cells includes for example using a stem cell bank. For example, using a stem cell bank includes thawing and expanding the ELA cells in a suitable medium, for example a RPMI 1640 medium or a RPMI 1680 medium. In various embodiments the ELA cells are expanded with a medium containing serum, for example 10% fetal bovine serum. In various embodiments, obtaining the populations includes collecting ELA stem cells from bodily fluids or tissues, and expanding the ELA stem cells in media.

An aspect of the invention provides a patient-specific master cell bank obtained by methods described herein. For example, the method of obtaining the population of ELA cells includes isolating the ELA cells and extending/expanding the ELA cells by culture prior to loading and administration to the subject.

An aspect of the invention provides a kit which includes: a single dose cell-based preparation of loaded ELA stem cells described herein, appropriate sterile packaging, and instructions for use. In various embodiments, the kit further comprises a therapeutic agent, a diagnostic agent, or a contrast agent. For example, the therapeutic agent is selected from: anti-coagulant, anti-tumor, anti-viral, anti-bacterial, anti-mycobacterial, anti-fungal, and anti-proliferative.

An aspect of the invention provides a method of manufacture for a cell-based preparation including: obtaining a population of ELA stem cells, and culturing the cells under conditions suitable to permit uptake by the cells of a diagnostic agent and a therapeutic agent. In various embodiments of the method, the diagnostic agent and/or the therapeutic agents includes Gadolinium-157 and/or Boron-10.

An aspect of the invention provides a cell bank of ELA cell lines each specific to a patient, such that populations of the ELA cells are obtained from the patient, and are isolated and are expanded by culture to obtain a patient-specific ELA cell bank.

### BRIEF DESCRIPTION OF DRAWINGS

FIG. 1 is a set of microphotographs showing ELA stem cells labeled with Molday ION Rhodamine B (MIRB). The incorporation of a diagnostic agent into the ELA stem cells confers diagnostic properties on the cell, allowing the cell to identify primary and metastic tumor locations. The left panel shows cells illuminated by phase contrast, the middle panel shows cells labeled with MIRB and the right panel shows a superimposed image of cells illuminated by phase contrast and cells labeled with MIRB.

### DETAILED DESCRIPTION

Early lineage adult stem cells (ELA) are adult progenitor cells that have extravasational and tissue regenerative properties. ELA stem cells are capable of differentiating into tissues of endodermal, mesodermal and ectodermal origin The source, isolation, characterization and certain uses and formulations of ELA cells are described in patent applications: U.S. provisional application Ser. Nos.: 60/927,596, 61/247,236, 61/247,242, 61/249,172, and 61/501,846, as well as U.S. patent application Ser. No. 12/598,047 and PCT applications serial number PCT/US2008/005742 and PCT/US2010/050288. These are each incorporated herein by reference in their entirety. Generally, the ELA cells are described by their expression of stem cell specific pluripotency genes (e.g., Oct-4, KFL-4, Nanog, Sox-2, Rex-1, GDF-3 and Stella). However, these cells are distinct from embryonic stem cells (ESC) and other types of early lineage adult cells such as MSC, VSEL, MAPC and cord blood derived progenitor cells because the ELA stem cells do not appear to detectibly express the various stem cell markers CD34, CD44, CD45, CD49a, CD66A, CD73, CD90, CD105, CSCR4, SSEA, or MHC class I or MHC class II structures, found on the above cell types. However, the ELA cells like ESCs, can form clusters or islands when grown under nonadherent conditions.

For the purposes of this disclosure, ELA cells are may be derived from multiple sources including, bone marrow stroma, blood, dermis, periosteum and tissues, but a useful exemplary source is synovial fluid (SF). The above patent applications detail the manner in which ELA cells can be obtained and propagated. In various embodiments, ELA cells are extracted from the patient intended to receive the cellular therapy (i.e., an autologous transplant). These ELA cells can be culture expanded prior to their introduction into the patient. Growth of ELA cells in vitro can be used, for example, to increase the number of ELA cells available for implantation or injection. In non-limiting example, ELA cell numbers can be increased about two fold or greater, about ten fold or greater, or about twenty fold or greater or more, depending on the desired number of cells. Patient-specific master cell banks, for example can comprise ten or more passages of expanded autologous cells, which increases ELA cell counts by many orders of magnitude in comparison to primary isolates. In various configurations, growing ELA cells in vitro can comprise expansion in a cell culture medium which comprises nutrients, buffers, salts, proteins, vitamins and/or growth factors, which promote ELA cell growth. A useful cell culture medium is RPMI 1680 supplemented with 10% serum, and appropriate antibiotics such as penicillin/streptomycin and G418. Human serum is preferred for expanding the ELA cells for human transplant preparations, but fetal bovine serum has produced acceptable yields of the ELA cells in culture. The expanded cells are commonly but not necessarily frozen prior to implantation, and a suitable cryogenic medium that is acceptable to the FDA for such purposes is CryoStorCS10, available from BioLife Solutions, Bothell, Wash.

For the following purposes of tracking and treating neoplastic tissues, either fresh isolates, expanded populations or cryopreserved populations can be used; likewise either as autologous, syngeneic or allograft preparations. The extravasational properties of adult stem cells facilitate the use of ELA cells in diagnostic applications. Cancers and neoplastic tissues, even benign tumors, cause the localized environment to generate inflammatory responses.

ELA stem cells will track to the sites of tissue injury, e.g., to neoplastic tissues. Accordingly, we describe herein, a population of culture expanded ELA stem cells, that are carriers of diagnostic agents, or carriers of both diagnostic and therapeutic agents. These are formulated and used for the treatment of neoplastic disorders, exemplified by brain, head and neck tumors, colon, breast, uterine, ovarian, lung, prostate, bone, pancreatic, gallbladder and liver cancers, and skin neoplasms particularly melanoma and its metastatic forms.

In various embodiments of the present invention, an ELA cell stem cell preparation having the diagnostic agent, or both diagnostic and therapeutic agents, is implanted or injected into a patient having a neoplastic disorder. The site and manner of implantation will vary with the type of location and type of the tumor, but essentially it is desirable to introduce the so prepared (i.e., “loaded”) ELA stem cells in close physical proximity or otherwise to enhance concentration of the loaded ELA cells at the neoplastic site. An exemplary preparation for injection can include sterile, isotonic, buffered saline (e.g., 0.9% NaCl) at an volume of 0.5 ml to 5 ml, containing approximately 10² to 10⁸ loaded ELA stem cells.

Also, another useful addition to the loaded ELA cell preparation is a perfluorocarbon agent such as perflourotributylamine. Perfluorates bind, transport and thereby increase the available oxygen at the treatment site. Emitted radiation can enhance the production of reactive oxygen species (ROS) at the treatment site, enhancing damage to neoplastic target tissue. Effective amounts of these perfluorate agents will be known to those having ordinary skill in the art, in view of the teachings provided herein. However, without limitation, exemplary effective amounts can range from 50 micrograms/ml to 1 milligram per ml in the cell culture media prior to loading the cells with the diagnostic and therapeutic agents. In addition, a radiocontrast agent, such as barium sulfate, or a radiocontrast dye, such as HYPAQUE may be included to aid the surgeon in tracking the movement and/or location of the injected material. Other suitable radiocontrast materials appropriate for use are known to persons skilled in the art. Other additives to provide surgical benefit may be included. Such additives include anesthetics, as palliative therapy or to reduce pain caused by the procedure, and antibiotics, to minimize the potential for bacterial infection. Treatment of the patient may further comprise treatment with radiosensitizing agents, such as capecitabine; or with other chemotheraphy and/or proton based radiation cycles; or other diagnostic imaging procedures.

### EXAMPLES

### Example 1

### Contrast Agent and Cell Labeling

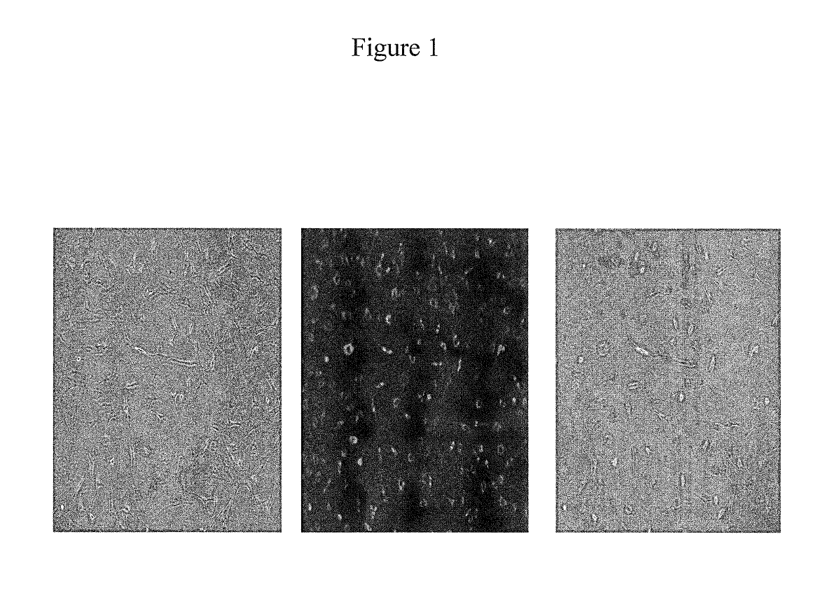

The contrast agents were incubated in sterilized 1.5 mL eppendorf tubes at room temperature (RT) for one hour to allow complete solubilization. Two contrast agents were employed to evaluate cell labeling, both provided by BioPAL, Inc.(Worcester, Mass.). The first contrast agent used was Molday ION Rhodamine (CL-50Q02-6A-50), a superparamagnetic iron oxide (SPIO) nanoparticle having a colloidal size of 50 nanometers (nm) and labeled with rhodamine B, a fluorescent dye, to allow visualization on both MRI and fluorescence. See FIG. 1. The second contrast agent is Gado CELL Track (BioPal Inc., catalog number CL-50P02-6), a Gd oxide (Gd₂O₃) nanoparticle having a colloidal size of 50 nm. To enhance cell uptake, the Gd₂O₃ nanoparticle is mixed with poly-L-lysine (PLL) (BioPAL Inc., catalog number CL-00-01).

Small volumes of Gd₂O₃ nanoparticles and PLL (2.5:1 ratio) are incubated in sterilized ELA cells and grown in 12-well plates (BD Falcon, Mississauga, ON, Canada) for two days in a 37° C., 5% CO₂ humidified incubator. When 70-80 percent confluence is reached, 1 mL of fresh media is added. ELA cell are incubated with Gd₂O₃ and returned to the incubator for 24 hours. The following concentrations are prepared for the incubation medium: 0, 0.002, 0.02, 0.1, and 0.2 mM of Gd; 0, 0.0036, 0.009, 0.018, and 0.036 mM of iron (Fe). Cells are washed three times with sterile lx PBS to remove excess contrast agents.

Fresh supplemented medium was then given to the cells (1 mL/well). The medium was changed every two to three days with subsequent washing, with the last wash carried out prior to MRI.

### Example 2

### Tracking ELA Stem Cell Migration and Treating Neoplasms

In accordance with one embodiment of the invention, the ELA stem cells are loaded with a diagnostic agent, thereby allowing tracking of the ELA stem cells to the site(s) of injured/neoplastic tissues. An ELA cell expressing a GFP conjugated transgene is described in our U.S. patent application Ser. No. 12/598,047. Detection of such a construct in tissues will be apparent to those skilled in the art. However, for the present invention, it is preferred to use Magnetic Resonance Imaging (MRI) to obtain detailed 3D images of the tissues, prior to, or following further treatment. MRI tracking of stem cells is described in the medical literature, such as to monitor stem cell migration after injection. For example administration of a gadolinium rhodamine dextran contrast agent, which can be resolved by both MRI and fluorescence microscopy, is described in Modo M, Cash D, Mellodew K, et al. Tracking Transplanted Stem Cell Migration Using Bifunctional, Contrast Agent-Enhanced, Magnetic Resonance Imaging. NeuroImage 2002; 17: 803-811. A similar preparation is Molday ION Rhodamine B-Labeled. ELA stem cells prepared by methods herein, shown in FIG. 1. This agent is available commercially from BioPal, Inc., and labeling of the stem cells followed manufacturers protocols for cell staining.

Boron neutron capture therapy (BNCT) is based on nuclear capture and fission reactions that occur when non-radioactive boron-10 (10B), is irradiated with neutrons of the appropriate energy to yield high energy alpha particles and reactive high energy lithium-7 (7Li) nuclei. However, the effectiveness of BNCT is dependent upon a relatively homogeneous uptake and distribution of 10 B within the tumor. Since healthy cells also uptake boron compounds, a tumor cell targeting means for a neutron capture agent such as through ELA cell delivery, provides a superior means than conventional nontargeted boron therapies. Following irradiation, both the alpha particles and the lithium ions produce ionizations with a range of approximately 5-9 microns, or approximately twice the diameter of an average ELA cell, so the therapeutic effect of ionizing radiation extends into the target cell.

In accordance with one embodiment of the invention, the ELA stem cells are loaded with a therapeutic agent, including a radiotherapeutic precursor such as a neutron capture agent. Examples of current clinically used therapeutic agents include the polyhedral borane anion, sodium borocaptate (BSII), and the dihydroxyboryl derivative of phenylalanine, boronophenylalanine (BPA). A more comprehensive review of low- and high-molecular-weight boron delivery agents that currently are being used or evaluated as potential boron sources for BNCT can be found at Wittig, A., Hideghety, K., Paquis, P., et al (2002). Sauerwein, W; Mass, R; Wittig, A, eds. “Current clinical results of the EORTC—study 11961”. Research and Development in Neutron Capture Therapy Proc. 10th Intl. Congress on Neutron Capture Therapy. pp. 1117-22.

Since the invention of magnetic resonance imaging (MRI), CT, neutron capture therapy, brachytherapy, and cell labeling technology, a parallel technology of injectable chemicals, referred to as contrast agents and neutron capture agents has developed. Contrast agents play an important role in the practice of medicine in that they help produce more useful images for diagnostic purposes. In the field of MRI, classes of imaging agents have been developed and adopted in clinical practice, including low molecular weight gadolinium complexes and colloidal iron oxides (U.S. Pat. No. 6,328,700 issued Dec. 11, 2001, which is incorporated by reference herein in its entirety). Gadolinium-based reagents (T1 agents) have the advantage of being brightening reagents, but have the disadvantage of having a short intravascular half-life. Colloidal iron oxide reagents (T2 agents) have the advantage of having a long intravascular half-life, but have the disadvantage of providing a weak brightening signal. Agents that can provide a long intravascular half-life and can provide a strong T1 signal (a brightening reagent) are highly desirable, combining the qualities of each of these classes of agents. Individual stable elements (e.g., gold) are known to have isotopes that are strongly detectable by neutron activation analysis. The known abundance of the “marker” elements in certain substances has permitted these elements to serve as markers for the substances. Sec Kennelly, J. J., et al. 1980; Can. J. Anim. Sci. 60:749-761. See also Nishiguchi Y., et al., 1996. Anim. Sci. Technol. (Jpn.), 67: 787-793.

Paramagnetic contrast agents and analogs labeled with stable isotopes are in various embodiments used with ELA stem cells. For example the paramagnetic contrast agents is a chelate of samarium (U.S. Pat. No. 7,048,907 issued May 23, 2006, which is incorporated by reference herein in its entirety). Examples of the latter are ¹⁵²Sm-diethylenetriaminepentaacetic acid Sm-DTPA, for example, ¹⁵²Sm-DTPA disodium or dimeglumine salts), ¹⁵²Sm-diethylenetriaminetriacetic acid bismethylamide (¹⁵²Sm-DTPA-BMA), and ¹⁵²Sm-tetraazacyclododecane tetraacetic acid (¹⁵²Sm-DOTA), for example, gadoteridol, Sm-hydroxypropyl tetraazacylo dodecanetriacetic acid (¹⁵²SmHP-DO3A). Similar compounds labeled with gadolinium (Gd) have been described, see, for example, in U.S. Pat. No. 4,885,363 issued Dec. 5, 1989, which is incorporated by reference herein in its entirety. The stable isotope labeled compound and/or the ELA stem cells may be administered to the subject by any suitable route, for example, a route that is intravenous, subcutaneous, intraperitoneal, intramuscular, or oral. Amounts employed should be suitable for detection, and may, for example, be about 5 μmol to about 1 mmol, or about 50 μmol to about 0.3 mmol, of agent per kg of body weight of the subject.

Gadolinium-157 (157Gd) a particularly advantageous dual purpose agent since it acts as both a neutron capture agent for radiotherapy, and since gadolinium compounds such as Gd-DTPA (gadopentetate dimeglumine Magnevisn, have been used routinely as contrast agents for magnetic resonance imaging (MRI), particularly in cancer diagnostic applications, their use as diagnostic agents are well known. Gamma rays, internal conversion and Auger electrons are products of the 157Gd (n,γ)158Gd capture reaction (157Gd+nth (0.025eV)→[158GD]→158Gd+γ+7.94 MeV). Gamma rays have long pathlengths, but the other radiation products (internal conversion and Auger electrons) have pathlengths of approximately 10 microns. Gadolinium and Iron based MRI imaging agents are also useful, and several varieties are commercially available from BioPal, Inc.

Following administration of the boron and or gadolinium-containing ELA stem cells, the tumor site is optionally imaged and then is irradiated with neutrons, e.g., by high energy (>0.5 eV<10 keV) epithermal neutron beams. BNCT is a highly selective type of radiation therapy that can selectively target the tumor at the cellular level without causing radiation damage to the adjacent normal cells and tissues. Doses up to 60-70 Gy can be delivered to the tumor cells in one or two applications compared to 6-7 weeks for conventional external beam photon irradiation. A more detailed review of dosimetry calculations and treatment modalities is provided by Coderre, J A; Morris, G M 1999. Radiation research 151 (1): 1-18. doi: 10.2307/3579742. PMID 9973079.In connection with the above embodiments, therapeutic outcome can be established numerous ways. Re-imaging the patient following neutron irradiation can reveal direct changes in tissue mass and morphology. Other therapeutic endpoints including palliative effects such as such as reduction in pain or symptoms; other endpoints of BNCT are well known in the art.

In various embodiments, the diagnostic agent or the therapeutic agent includes a stable isotope labeled compound (for example, xenobiotics). In various embodiments, the labeled compounds includes at least one of: a low molecular weight compounds, e.g., organic compounds (less than about 5000 kDa), and rare earth chelates and chelates of bile acids; a high molecular weight compounds (more than about 5000 kDa) such as chelates of polymers, such as polysaccharides, polypeptides including proteins, and polynucleotides including nucleic acids; and a colloid.

The invention in various embodiments now having been fully described, additional embodiments are exemplified by the following claims, which are not intended to be construed as further limiting. The contents of all cited references are hereby incorporated by reference herein in their entireties.

### ADDITIONAL REFERENCES

Aboody K S, Brown A, Rainov N G, Bower K A, Liu 5, Yang W et al. Neural stem cells display extensive tropism for pathology in adult brain: evidence from intracranial gliomas. Proc Natl Acad Sci USA 2000; 97: 12846-12851.

Dwyer RM, Potter-Beirne SM, Harrington Ka, et al. Monocyte Chemotactic Protein-1 Secreted by Primary Breast Tumors Stimulates Migration of Mesenchymal Stem Cells. Human Cancer Biology 2007; 13(17): 5020-5027.

Pereboeva L, Komarova 5, Mikheeva G, Krasnykh V, Curiel D T. Approaches to utilize mesenchymal progenitor cells as cellular vehicles. Stem Cells 2003; 21: 389-404.

Jiang Y, Jahagirdar B N, Reinhardt R L, et al. Pluripotency of mesenchymal stem cells derived from adult marrow. Nature 2002; 418: 41-49.

Belmadani A, Tran P B, Ren D, Miller R J. Chemokines Regulate the Migration of Neural Progenitors to Sites of Neuroinflammation. Journal of Neuroscience 2006; 16(12): 3182-3191.

Sun L, Lee J, Fine H A. Neuronally expressed stem cell factor induces neural stem cell migration to areas of brain injury. J Clin Invest 2004; 113: 1364-1374

Reese O, Disko A, Zirkel D, Westphal M, Lamszus K. Neural stem cell migration toward gliomas in vitro. Neuro Oncol 2005; 7: 476-484.

Francois S, Bensidhoum M, Mouiseddine M, et al. Local irradiation not only induces homing of human mesenchymal stem cells at exposed sites but promotes their widespread engraftment to multiple organs: a study of their quantitative distribution after irradiation damage. Stem Cells 2006; 24: 1020-29.

Najbauer J, Danks M K, Schmidt N O, Kim S U, Aboody KS . Neural stem cell-mediated therapy of primary and metastatic solid tumors. In: Bertolotti R, Ozawa K (eds). Progress in Gene Therapy, Autologous and Cancer Stem Cell Gene Therapy. World Scientific: Singapore, 2007.

Kim S K, Kim S U, Park I H, Bang J H, Aboody K S, Wang K C et al. Human neural stem cells target experimental intracranial medulloblastoma and deliver a therapeutic gene leading to tumor regression. Clin Cancer Res 2006; 12: 5550-5556.

Shimato S, Natsume A, Takeuchi H, Wakabayashi T, Fujii M, Ito M et al. Human neural stem cells target and deliver therapeutic gene to experimental leptomeningeal medulloblastoma. Gene Therapy 2007; 14: 1132-1142

Kim S K, Cargioli TG, Machluf M, Yang W, Sun Y, Al-Hashem R et al. PEX-producing human neural stem cells inhibit tumor growth in a mouse glioma model. Clin Cancer Res 2005; 11: 5965-5970.

Benedetti S, Pirola B, Polio B, et al. Gene therapy of experimental brain tumors using neural progenitor cells. Nat Med 2000; 6: 447-50.

Tang Y, Shah K, Messerli S M, Snyder E, Breakfield X, Weissleder R. In vivo tracking of neural progenitor cell migration to glioblastomas. Hum Gene Ther 2003; 14: 1247-54.

Shah K, Hingtgen S, Kasmieh R, et al. Bimodal Viral Vectors and In Vivo Imaging Reveal the Fate of Human Neural Stem Cells in Experimental Glioma Model. Journal of Neuroscience 2008; 28(17): 4406-4413.

Shah K, Bureau E, Kim D, et al. Glioma Therapy and Real-Time Imaging of Neural Precursor Cell Migration and Tumor Regression. Ann Neurol 2005; 57: 34-41.

Corsten M F, Shah K. Therapeutic stem-cells for cancer treatment: hopes and hurdles in tactical warfare. Lancer Oncol 2008; 9: 376-84

Cerullo, N.; Bufalino, D.; Daquino, G. (2009). “Progress in the use of gadolinium for NCT”. Applied Radiation and Isotopes 67 (7-8): S157-60. doi:10.1016/j.apradiso.2009.03.109. PMID 19410468.

Yasui, Linda S.; Andorf, Christine; Schneider, Linda; Kroc, Thomas; Lennox, Arlene; Saroja, K. R. (2008). “Gadolinium neutron capture in glioblastoma multiforme cells”. International Journal of Radiation Biology 84 (12): 1130-9. doi: 10.1080/09553000802538092. PMID 19061138.

Nemoto, Hisao; Cai, Jianping; Nakamura, Hiroyuki; Fujiwara, Masaru; Yamamoto, Yoshinori (1999). “The synthesis of a carborane gadolinium-DTPA complex for boron neutron capture therapy”. Journal of Organometallic Chemistry 581: 170-5. doi:10.1016/S0022-328X(99)00049-2.

Tokumitsu, Hiroyuki; Hiratsuka, Junichi; Sakurai, Yoshinori; Kobayashi, Tooru; Ichikawa, Hideki; Fukumori, Yoshinobu (2000). “Gadolinium neutron-capture therapy using novel gadopentetic acid-chitosan complex nanoparticles: In vivo growth suppression of experimental melanoma solid tumor”. Cancer Letters 150 (2): 177-82. doi:10.1016/S0304-3835(99)00388-2. PMID 10704740.

De Stasio, G.; Mesh, D; Ford, J M; Daniels, M J; Erhardt, RJ; Frazer, BH; Tyliszczak, T; Gilles, M K et al. (2006). “Motexafin-Gadolinium Taken Up in vitro by at Least 90% of Glioblastoma Cell Nuclei”. Clinical Cancer Research 12 (1): 206-13. doi:10.1158/1078-0432.CCR-05-0743. PMID 16397044.

Geninatti-Crich, Simonetta; Alberti, Diego; Szabo, Ibolya; Deagostino, Annamaria; Toppino, Antonio; Barge, Alessandro; Ballarini, Francesca; Bortolussi, Silva et al. (2011). “MRI-Guided Neutron Capture Therapy by Use of a Dual Gadolinium/Boron Agent Targeted at Tumour Cells through Upregulated Low-Density Lipoprotein Transporters”. Chemistry 17 (30): 8479-86. doi:10.1002/chem.201003741. PMID 21671294.

What is claimed is:

1. A cell-based preparation comprising: a population of loaded ELA stem cells further comprising a diagnostic agent and a therapeutic agent.

2. The cell-based preparation of claim 1, wherein the diagnostic agent is Gadolinium-157.

3. The cell-based preparation of claim 1, wherein the therapeutic agent is selected from at least one of Gadolinium-157 and Boron-10.

4. The cell-based preparation of claim 1, wherein the loaded ELA stem cells are in an amount of 10² to 10⁸.

5. The cell-based preparation of claim 4, wherein the loaded ELA stem cells are in a volume of 0.5 to 10 ml.

6. The cell-based preparation of claim 4, further comprising a pharmaceutically acceptable solution.

7. The cell-based preparation of claim 1, further comprising a perfluorate agent.

8. The cell-based preparation of claim 4, further comprising unloaded ELA stem cells, wherein the ratio of the loaded cells to the unloaded cells is greater than 2:1.

9. The cell-based preparation of claim 8, wherein the ratio of the loaded cells to the unloaded cells is greater than 5:1.

10. A method of treating a subject having a cancer or a neoplasm, the method comprising:
introducing to the subject a quantity of the cell-based preparation of claim 1 or claim 4 which targets to the neoplasm, and
administering radiotherapy by exposing the subject to a neutron source of greater than 0.5 eV to about 10 keV, thereby treating the neoplasm of the subject by the radiotherapy.

11. The method according to claim 10, further comprising prior to exposing, imaging the subject.

12. The method of claim 10, further comprising prior to introducing, obtaining from the subject a population of ELA cells, isolating the ELA cells and extending the ELA cells by culture prior to loading and administration to the subject.

13. A patient-specific master cell bank prepared by the method of claim 12 prior to introducing to the subject the cell-based preparation.

14. A kit comprising: a single dose cell-based preparation of loaded ELA stem cells of claim 1, appropriate sterile packaging, and instructions for use.

15. A method of manufacture for a cell-based preparation comprising: obtaining a population of ELA stem cells, and culturing the cells under conditions suitable to permit uptake by the cells of a diagnostic agent and a therapeutic agent.

16. The method of claim 15, wherein the diagnostic and therapeutic agents is selected from at least one of Gadolinium-157 and Boron-10.

17. A cell bank of ELA cell lines each specific to a patient, wherein populations of the ELA cells are obtained from the patient, and are isolated and are expanded by culture to obtain a patient-specific ELA cell bank.
